(19) United States
(12) Patent Application Publication
(10) Pub. No.: US 2025/0303020 A1
(43) Pub. Date: Oct. 2, 2025

(54) PH-RESPONSIVE HYDROGEL BIOCARRIER AND APPLICATION

(71) Applicant: BIO-BANK CORP., Shandong (CN)

(72) Inventors: Xinghui Zhao, Shandong (CN); Junhui Zhai, Shandong (CN); Hong Du, Shandong (CN); Kelong Wang, Shandong (CN)

(73) Assignee: BIO-BANK CORP., Shandong (CN)

(21) Appl. No.: 18/705,736

(22) PCT Filed: Mar. 7, 2022
(86) PCT No.: PCT/CN2022/079518

§ 371 Date: Oct. 8, 2024

(30) Foreign Application Priority Data
Nov. 3, 2021 (CN) 202111296151.2

(51) Int. Cl. A61L 26/00; C08F 2/50; C08F 251/00

(52) U.S. Cl. CPC A61L 26/008; A61L 26/0066; C08F 2/50; C08F 251/00; A61L 2300/414; A61L 2430/34

(57) ABSTRACT

Disclosed in the present invention is a pH-responsive hydrogel biocarrier. The pH-responsive hydrogel biocarrier is prepared by irradiating sodium hyaluronate and methacrylic anhydride by using 365 nm ultraviolet laser in the presence of a photoinitiator, and hydroxyl on a side chain of sodium hyaluronate is polymerized into a methacrylate derivative of hyaluronic acid by means of photocrosslinking and esterification. Also disclosed in the present invention is a pH-responsive hydrogel biocarrier loaded with a therapeutic protein PDGF-BB. In the present invention, an in-situ free radical polymerization method is used for synthesis, a reaction condition is mild, and biological activity of PDGF-BB is not influenced; and PDGF-BB is wrapped in the hydrogel, preventing the expose in an enzyme environment, and improving the stability of the biological activity of PDGF-BB.

### TECHNICAL FIELD

The disclosure discloses a hydrogel biocarrier, which belongs to the technical field of pharmacy.

### BACKGROUND ART

Platelet-derived growth factor (PDGF) is a cationic glycoprotein that is heat-resistant, acid resistant, and easily hydrolyzed by proteases. It can be secreted by various cells, such as platelets, fibroblasts, macrophages, and so on. PDGF has four monomers: PDGF-A, PDGF-B, PDGF-C, and PDGF-D, which are connected by disulfide bonds to form dimeric forms. Among them, PDGF-BB, the dimeric form of PDGF-B, as a mitotic promoter, can stimulate the proliferation and migration of fibroblasts and smooth muscle cells, promote macrophage production and secretion of growth factors. The role is more significant in wound repair, tissue regeneration, bone and tooth regeneration, and joint repair. Especially in wound repair, it is the only growth factor approved by the US Food and Drug Administration (FDA) for the treatment of diabetes ulcers.

### SUMMARY

### Technical Problems

Wound repair and tissue regeneration diseases require growth factors to maintain a certain concentration in the affected area for a long time. However, PDGF-BB has a short half-life (<2 minutes) and is easily degraded by proteases. In chronic wound beds, there are large amounts of proteases, making PDGF-BB more prone to inactivation. Usually, multiple high doses are administered to maintain drug concentration, but there is a risk of tumorigenesis. So it requires a carrier that can improve the stability of PDGF-BB while maintaining a certain drug concentration for a long time to ensure the safety and effectiveness of PDGF medication in this field.

### Technical Solutions

Based on the above technical problems existing in the field, the purpose of the disclosure is to provide a hydrogel biocarrier, which can not only release the biological activity of PDGF-BB in a controllable manner, improve its stability in pharmacodynamics, but also ensure the biological safety of drug use through the degradation of the carrier.

The biocompatibility and the capacity of ensuring the slow release of growth factors of methacrylic anhydride hyaluronic acid hydrogel are considered comprehensively in this disclosure. By introducing PDGF-BB into the methacrylic anhydride hyaluronic acid hydrogel network, the pH-responsive hydrogel biocarrier combined with growth factors is prepared, which aims to improve the biocompatibility, pH responsiveness, detachment and protection of cell layer, as well as slow release of growth factors.

Based on the above purpose, the disclosure first provides a pH-responsive hydrogel biocarrier, which is prepared by crosslinking reaction of methacrylated hyaluronic acid which is initiated by irradiation of 365 nm ultraviolet laser in the presence of photoinitiator and crosslinker.

In an optional embodiment, the photoinitiator is I2959, and the crosslinker is a first crosslinker GDMA and a second crosslinker AI102.

In another optional embodiment, the methacrylated hyaluronic acid is synthesized from esterification of sodium hyaluronate and methacrylic anhydride in a molar ratio of 1:30.

In an optional embodiment, the molar ratio of the first crosslinker GDMA to the second crosslinker AI102 is (1-4):(0-2).

Secondly, the disclosure provides a method of preparing the pH-responsive hydrogel biocarrier. The method includes that methacrylated hyaluronic acid is initiated into a crosslinking reaction which is irradiated by a 365 nm ultraviolet laser in the presence of a photoinitiator, a first crosslinker GDMA and a second crosslinker AI102.

Thirdly, the disclosure provides a pH-responsive hydrogel biocarrier loaded with therapeutic protein. The pH-responsive hydrogel biocarrier loaded with therapeutic protein is prepared by freeze-drying the hydrogel biocarrier and loading the therapeutic protein into the pH-responsive hydrogel biocarrier by dry immersion method.

In an optional embodiment, the therapeutic protein is platelet-derived growth factor.

In a more optional embodiment, the platelet-derived growth factor is in the form of BB type dimer.

Fourthly, the disclosure provides a method of preparing the pH-responsive hydrogel biocarrier loaded with therapeutic protein, which includes the steps of freeze-drying the hydrogel biocarrier, and loading the therapeutic protein into the pH-responsive hydrogel biocarrier by the dry immersion method.

Finally, the disclosure provides the application of the pH-responsive hydrogel biocarrier loaded with therapeutic protein in the preparation of drugs for promoting wound repair and tissue regeneration.

### Beneficial Effects of the Present Disclosure

The disclosure provides a method of in-situ free radical polymerization to synthesize pH-responsive hydrogel biocarrier, and uses the dry immersion method to load the therapeutic protein into the pH-responsive hydrogel biocarrier. The reaction conditions are mild, and the biological activity of PDGF-BB is not affected; Wrapping PDGF-BB in hydrogel can avoid exposure to enzyme environment and the stability is improved. The release of PDGF-BB is achieved through the degradation of hydrogel. Two crosslinkers. GDMA (crosslinker 1) and AI102 (crosslinker 2) whose degradation are responsive to the alkaline pH, are selected in the disclosure. The said degradation of the two crosslinkers is based on the ester bond groups of themself, but the degradation rate of the two crosslinkers is different under alkaline conditions. To meet the application requirements, the degradation rate of the gel can be controlled by regulating the compatibility ratio of the pH-responsive hydrogel biocarrier provided by the disclosure according to different wound types, that is, different pH in different wound type. Given that the pH of the human wound site is around 8.0, the degradation rate of the two crosslinkers is different at pH 8.0. Crosslinker 2 has better water solubility than crosslinker 1 and contains relatively more ester bonds, so the degradation rate of crosslinker 2 is faster than that of crosslinker 1. By adjusting the ratio of the two crosslinker, for example, when the ratio of GDMA:AI102 is 1:2, the release rate is the slowest, so that the ratio between the two crosslinkers can be controlled to achieve the degradation of hydrogels at different rates. Suitable crosslinker ratios can be selected for the treatment of different diseases and wound types (see FIG. 1).

### BRIEF DESCRIPTION OF THE DRAWINGS

FIG. 1: Schematic diagram of the preparation of PDGF-BB pH responsive hydrogel with controlled release;

FIG. 2: m-HA ¹H-NMR spectrum;

FIG. 3: Physical picture of controllable-released PDGF-BB pH responsive hydrogel:

FIG. 4: Compression performance test of pH responsive hydrogels with different crosslinker ratios;

FIG. 5: Scanning electron microscopy of pH responsive hydrogels with different crosslinker ratios;

FIG. 6: Diagram of pH responsive hydrogel swelling rate with different crosslinker ratios;

FIG. 7: Diagram of Cumulative release rate of PDGF-BB from pH responsive hydrogels with different crosslinker ratios;

FIG. 8: The cytotoxicity test of pH responsive hydrogel;

FIG. 9: Statistical diagram of migration effect of pH responsive hydrogel on 3T3 cells;

FIG. 10. Observation of the migration effect of pH responsive hydrogel on 3T3 cells;

FIG. 11. Observation of wound healing in full layer skin of mice treated with different modes;

FIG. 12. The time-based varying curves of the wound area of the full layer skin in the mice treated with different mode.

### EXAMPLES

The following will further describe the present disclosure in conjunction with specific embodiments, and the advantages and features of the present disclosure will become clearer as described. But these embodiments are only exemplary and do not constitute any limitation on the scope of protection limited by the claims of the present disclosure.

### Example 1. Preparation of pH-Responsive Hydrogel Biocarrier

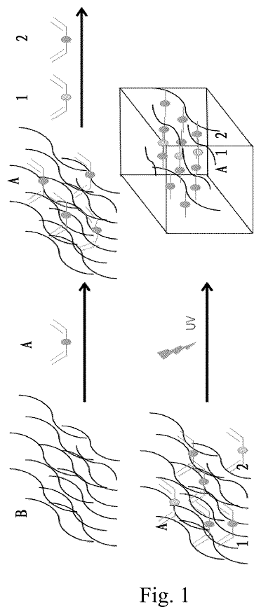

FIG. 1 shows the schematic diagram of the preparation of PDGF-BB pH responsive hydrogel, where A is methacrylic anhydride, B is sodium hyaluronate, 1 is a first crosslinker GDMA, and 2 is a second crosslinker AI102. The following are specific preparation steps.

1. Preparation of Methacrylated Hyaluronic Acid (m-HA)

Sodium hyaluronate (HA) and methacrylic anhydride (MA) are esterified to synthesize methacrylic acid derivatives of hyaluronic acid. The specific steps include the following.
(1) Weigh 2 g of sodium hyaluronate (HA) powder and dissolve it in 100 mL of deionized water. Stir overnight in a refrigerator at 4° C. to fully dissolve the powder.
(2) Adjust the pH of the HA solution by adding 5 mol/L sodium hydroxide to maintain the reaction pH between 8 and 9. Then slowly add 1.6 mL of methacrylic anhydride to the HA solution and stir continuously at 4° C. for 24 hours.
(3) Subsequently, m-HA was precipitated in excess acetone, washed with ethanol, and then dissolved in 50 ml of deionized water.
(4) A dialysis bag with a molecular weight cut-off of 14000 Da was used to dialyze with deionized water for 48 hours to remove unreacted methacrylic anhydride and other by-products, obtaining purified m-HA, which was freeze-dried for later use.

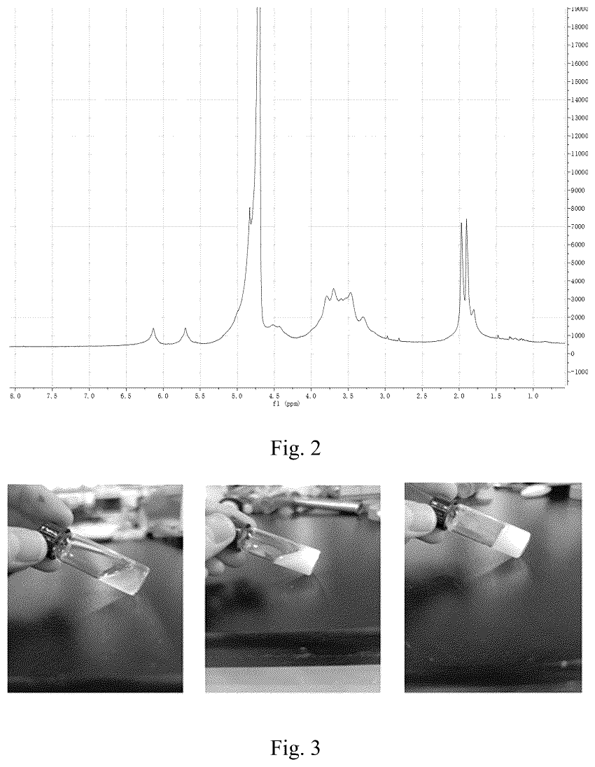

Dissolve m-HA in deuterated water as a solvent at a concentration of 6 mg/ml for nuclear magnetic resonance characterization. The m-HA nuclear magnetic resonance image is shown in FIG. 2. From the ¹H-NMR spectrum, the characteristic peak of D₂O can be seen at δ 4.79 ppm; the characteristic peak of proton hydrogen in the HA ring structure can be seen at δ 3-4 ppm; the characteristic peak of H on the methyl group in HA can be seen at δ 1.86 ppm; the characteristic peak of methyl H on the side chain N-acetylglucosamine can be seen at δ 2.03 ppm. Compared with the HA nuclear magnetic resonance spectrum, m-HA exhibits two new peaks of δ 5.68 ppm and δ 6.13 ppm which are the characteristic peaks of olefin protons in methacrylic anhydride, indicating that methacrylic anhydride has successfully been grafted into the HA molecular structure.

2. Preparation of pH-Responsive Hydrogel Biocarrier
Preparation of m-HA Hydrogel Loaded with Platelet-Derived Growth Factor

The pH responsive hydrogel biocarrier was prepared with m-HA as monomer, GDMA (glyceryl dimethacrylate, crosslinker 1) and AI102 (polylactic acid polyethylene glycol polylactic acid acrylate, crosslinker 2) as crosslinkers, and then initiator I2959 (2-hydroxy-4′-(2-hydroxyethoxy)-2-methylphenylacetone) was added to initiate the reaction at 365 nm of ultraviolet light, and then freeze-dried.

The specific steps are as follows:
Prepare an aqueous solution of methacrylated hyaluronic acid and the second crosslinker AI102: The first crosslinker GDMA and initiator I2959 were added to DMSO to prepare solutions, respectively;
Mix m-HA, AI102 aqueous solution. I2959 solution and GDMA solution evenly in the reaction tube, and react under 365 nm ultraviolet light for 2 min to form pH-responsive hydrogel biocarrier. After freeze-drying, PDGF-BB was loaded into the hydrogel.

The molar ratio of the first crosslinker GDMA to the second crosslinker AI102 is (1-4): (0-2);

### (1) Solution Preparation:

Take 20 mg of freeze-dried m-HA and add 1 ml of deionized water to prepare a solution of 20 mg/ml;
Take 50 mg of AI102 and add 500 μl of deionized water. Prepare a solution of 100 mg/ml;
Take 250 mg of GDMA and add DMSO 500 μl. Prepare a solution of 500 mg/ml;
Take 100 mg of I2959 and add 1 ml of DMSO to prepare a solution of 100 mg/ml.
   (2) Preparation of Hydrogels with Different Crosslinker Ratios:

TABLE 1
Hydrogel synthesis parameters (crosslinker ratio of GDMA:AI102)

| Crosslinker | | | | |
|---|---|---|---|---|
| ratio | PDGF-BB | GDMA | AI102 | I2959 |
| 1:0 | 1 | 600 | 0 | 480 |
| 4:1 | 1 | 480 | 120 | 480 |
| 1:1 | 1 | 300 | 300 | 480 |
| 1:2 | 1 | 200 | 400 | 480 |

Mix m-HA, AI102 aqueous solution, I2959 solution and GDMA solution in a reaction tube, and react for 2 min under 365 nm ultraviolet light to obtain hydrogels of different proportions.

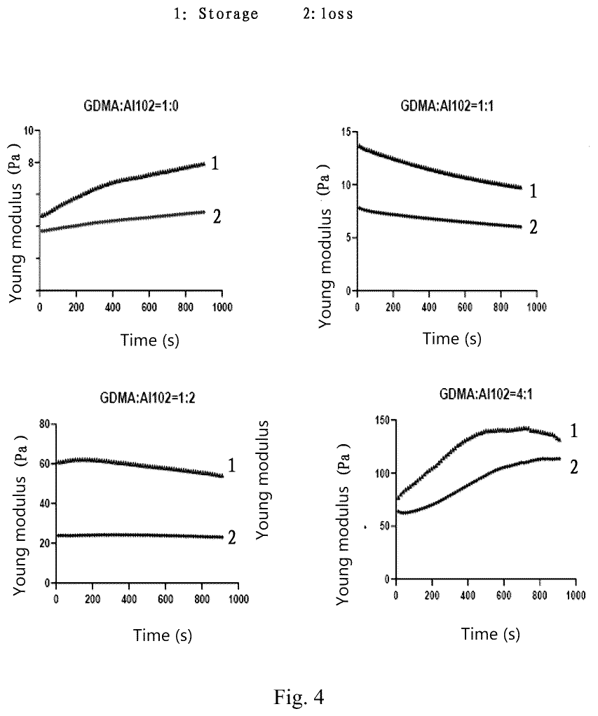

FIG. 3 shows the physical picture of PDGF-BB pH responsive hydrogel with controlled release. The compression performance test results of pH responsive hydrogels with different crosslinker ratios are shown in FIG. 4. As the crosslinking dose increases, the Young's modulus significantly increases. The larger the Young's modulus, the less likely the material is to undergo deformation. At the same compression strain, the more crosslinker, the higher the stress required by the hydrogel, and the higher the compression modulus. From the results, it can be seen that when GDMA:AI102=4:1, the Young's modulus is maximum, indicating the minimum deformation under stress.

3. PDGF-BB Loading into pH-Responsive Hydrogel Biocarrier

PDGF-BB was loaded into pH-responsive hydrogel biocarrier by dry immersion method, that is, when the hydrogel was completely freeze-dried, it was immersed in PDGF-BB solution to fully load PDGF-BB into the hydrogel. The reaction is completed to form a hydrogel containing PDGF-BB.

The specific steps are as follows:

The hydrogel was drilled and sampled with a hole punch to make a cylinder with a diameter of 15 mm and a height of 8 mm. Then the hydrogel was freeze-dried for 3 days, and then the freeze-dried hydrogel was immersed in 5 ml of 50 ng/md PDGF-BB solution for 48 h to ensure that the hydrogel was fully absorbed. Then, it was washed three times with PBS, and the PDGF-BB that was not bound to the hydrogel was washed away to prepare a hydrogel biocarrier that combined with growth factors.

### Example 2. Characterization of m-HA Hydrogel Loaded with Platelet-Derived Growth Factor

### 1. Characterization of Hydrogel Morphology

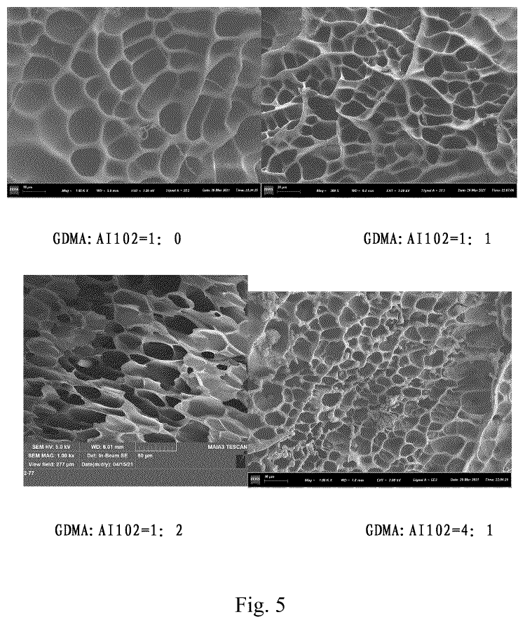

The hydrogel sample that has reached equilibrium swelling was freeze-dried in a lyophilizer at −50° C. for 48 h. The obtained sample was prepared by spraying gold after freezing and brittle fracture in liquid nitrogen, and its cross-sectional morphology was observed by scanning electron microscope (SEM). FIG. 5 shows the scanning electron microscope images of pH responsive hydrogels with different crosslinker ratios. It can be seen that the larger the proportion of crosslinker and the smaller the pore size, indicating the higher the degree of crosslinking. When the molar ratio of GDMA to AI102 is 1:2, the degree of crosslinking is highest.

### 2. Characterization of Swelling Ratio of Hydrogel

The freeze-dried hydrogel scaffold was used to test the swelling performance. Punch the hydrogel sample with a punch to make a cylinder with a diameter of 1.5 cm and a height of 12 mm, and then freeze dry the sample. Use the analytical balance to weigh and record the original weight W₀ of each group of hydrogels, and then put the hydrogels into 10 mL PBS buffer solution to soak, observe the swelling change of hydrogels, and take samples at 2 h, 6 h, 12 h, 24 h, 48 h and 72 h respectively. Carefully absorb the water on the surface of the hydrogel with filter paper, weigh and record the weight Wᵢ of the hydrogel sample, set at least 3 duplicate samples for each sample, and take the average of the final results. The swelling ratio (W) of hydrogel can be calculated by the following formula:

$$Swelling rate ( W ) = ( {W}_{i} - {W}_{0} ) / {W}_{0} \times 100 \%$$

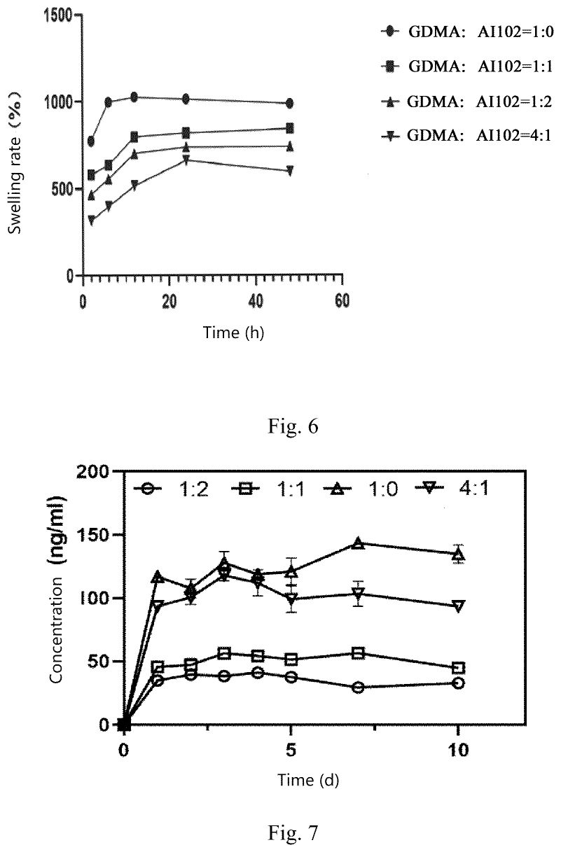

As shown in FIG. 6, the swelling rate is highest when the GDMA:AI102 ratio is 1:0. When the ratio of the two is 4:1, the swelling rate is the smallest. In the first 14 hours, the swelling degree of the hydrogel scaffold rose sharply, especially for the two kinds of hydrogels with less crosslinker dosage. The main reason is that the amount of crosslinker added is less, and the cross-linking degree is low, then the polymer network formed is relatively scattered, which can absorb a lot of water. The hydrogel scaffold reached swelling equilibrium after soaking for about 24 hours.

3. Characterization of pH Responsive Hydrogel Release Kinetics with Different Crosslinker Ratios

Dry HA-MA hydrogels loaded with PDGF-BB with different crosslinker ratios were placed in PBS buffer solution with pH8, incubated in a 37° C. water bath, and drug controlled release was studied. Retrieve 200 μL of release liquid at specific times (1, 2, 3, 4, 5, 7, 10 days) and replenish 200 μL of pure PBS buffer solution at the same time. Measure the content of PDGF-BB released from the collected release solution using enzyme-linked immunosorbent assay (ELISA).

The cumulative release rate of PDGF-BB from pH responsive hydrogels with different crosslinker ratios is shown in FIG. 7. When the GDMA:AI102 ratio is 1:0, the release rate is the fastest. When the ratio of the two is 1:2, the release rate is the slowest.

### 4. Biocompatibility Characterization of pH Responsive Hydrogel

### Cytotoxicity Testing:

Take the prepared hydrogel without PDGF-BB and the hydrogel with PDGF-BB, add an appropriate amount to the 24 well plate, and add an appropriate amount of P/S only medium (pH=8). After continuing incubation in the incubator for 24 hours, take out the medium, and filtered through 0.22 μm filter membrane. Inoculate 1×10⁴ cells in each well and incubate overnight at 37° C. and 5% CO₂. Add the above extract and incubate for 24 hours, then use CCK-8 kit to verify whether the hydrogel has cytotoxicity. The ratio of crosslinker 1 to crosslinker 2 of the hydrogel used is 1:2.

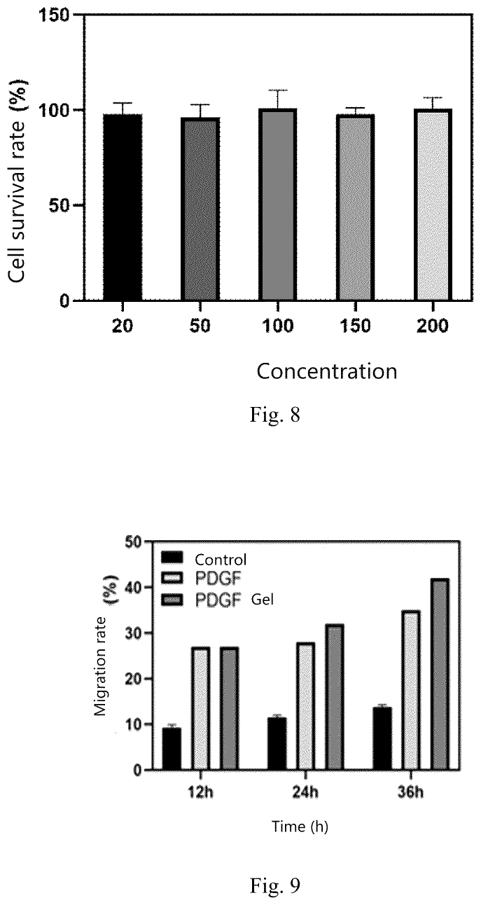

The cytotoxicity test results of pH responsive hydrogel are shown in FIG. 8. The bioactivity of the hydrogels coated with different concentrations of PDGF-BB is more than 75%, so it is considered that the prepared hydrogels have good biocompatibility.

### 5. Migration Effect of pH Responsive Hydrogel on 3T3 Cells

(1) Preparation of serum-free DMEM medium (pH=8): Take 0.5 ml of dual antibiotics and then add 49.5 ml of DMEM high glucose medium.
(2) Place the ruler and marker pen on the clean bench for UV sterilization for 30 minutes, then incubate DMEM complete medium with 1×PBS in a 37° C. water bath for 20 minutes, and incubate trypsin at room temperature.
(3) Plating: Take a 6-well plate and use a marker to draw 5 horizontal lines through the well on the back of the plate. Dilute the cells to 5×10⁴ cells/ml and inoculate 2 ml into each well. Incubate under the condition of 37° C. and 5% CO₂, so that the bottom of the well in the plate is covered with monolayer cells.
(4) In order to eliminate the impact of complete culture medium on cell proliferation and migration, 12 hours before sample addition, the complete medium was replaced with culture medium containing only P/S. Use 10 μL pipette tip drawing a straight line perpendicular to the marking line. Remove the old culture medium and rinse 3 times with 1×PBS to remove the scratched cells.
(5) The serum-free DMEM medium was used to soak the hydrogel without PDGF and the hydrogel containing PDGF-BB, respectively. Take 2 ml of the above mentioned extraction solution and add it to each well separately. Set up a blank group (containing only P/S medium). Add the sample and incubate it in a 5% CO₂ incubator at 37° C. Take photos at 0, 12, 24, 36, and 48 hours under the same field of view, calculate the area using Image J software, and then calculate the cell migration rate.

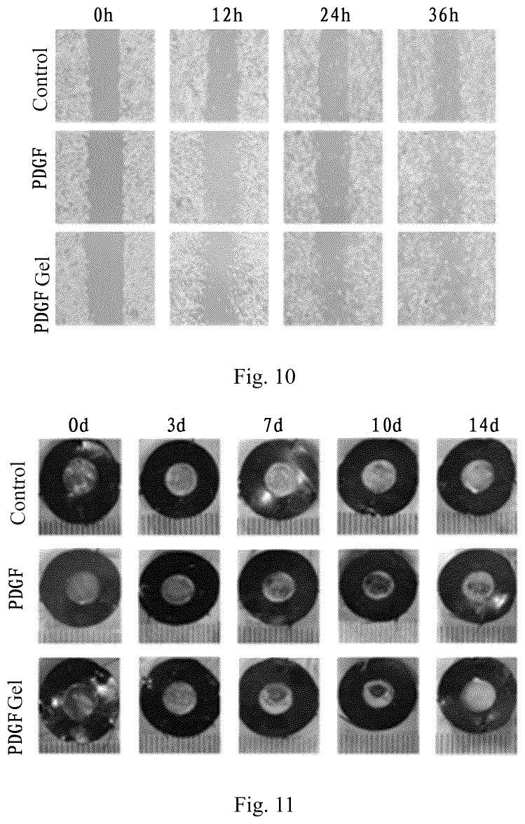

FIG. 9 is the statistical diagram of the migration effect of pH responsive hydrogel on 3T3 cells; FIG. 10 shows the observation of migration effect of pH responsive hydrogel on 3T3 cells. The ratio of crosslinker 1 to crosslinker 2 of the hydrogel used is 1:2. Compared with the control group containing only hydrogel, the 3T3 cells treated with uncoated PDGF-BB and hydrogel-coated PDGF-BB continuously migrated to the middle part, and the scratch area was significantly reduced. By quantifying the wound area with ImageJ, it was found that the cell migration rate of the uncoated PDGF-BB could reach 37% within 36 hours, the cell migration rate of the hydrogel-coated PDGF-BB could reach 42% within 36 hours, while the cell migration rate of the control group was only about 10%. This proves that the hydrogel-coated PDGF-BB can promote 3T3 cell migration, which may promote wound healing.

### 6. Full-Thickness Skin Wound Healing Experiment in Mice:

Mice were divided into three groups: control, hydrogel without PDGF-BB and hydrogel with PDGF-BB. The ratio of crosslinker 1 to crosslinker 2 of the hydrogel is 1:2. The drug-loaded hydrogel was injected into the wound site in situ (pH=8), and then covered with non-adhesive sterile dressing, and fixed with 3M dressing. Take photos of mouse wounds at different time points (0, 3, 7, 10, 14 days), using a ruler as a reference. The wound area was calculated using Image J software.

Wound closure rate %=(S₀−S_{N})/S₀×100, wherein, S₀ is the initial wound area, and S_{N} is the wound area at N days.

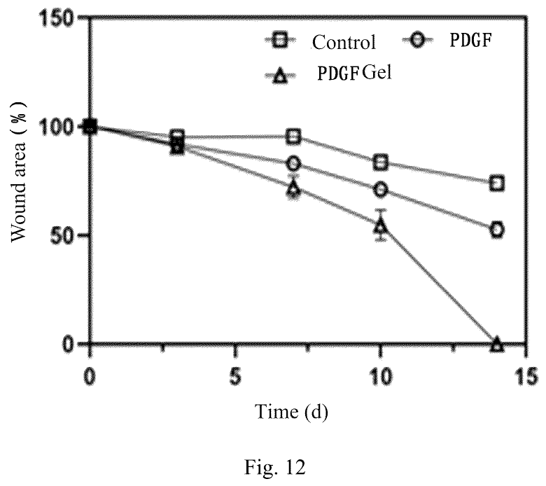

FIG. 1 is a graphical representation of the healing of full-thickness skin wounds in mice treated with different modes. It can be seen from FIG. 1I that the wounds of diabetes mice are gradually shrinking in all three groups, but the wound healing of the hydrogel-coated PDGF-BB group is more significant than that of the other two groups. The time-dependent curve of mouse wound area in FIG. 12 shows that at 7 days, the average wound areas of the three groups were 83.4%, 72.8%, and 65.0%, respectively; On the 14th day, the average wound area of the three groups became 54.9, 25.1% and 7.8%. Compared with the other two groups, the wound area of the hydrogel-coated PDGF-BB group decreased the most. The results indicated that the hydrogel-coated PDGF-BB group is conducive to wound healing.

### INDUSTRIAL APPLICABILITY

The disclosure discloses a pH responsive hydrogel. The pH responsive hydrogel is easy to be prepared industrially, showing the industrial applicability.

1. A pH-responsive hydrogel biocarrier, wherein the pH-responsive hydrogel biocarrier is prepared by crosslinking reaction of methacrylated hyaluronic acid which is initiated by irradiation of 365 nm ultraviolet laser in the presence of a photoinitiator and crosslinker.

2. The pH-responsive hydrogel biocarrier according to claim 1, wherein the photoinitiator is I2959, and the crosslinker is a first crosslinker GDMA and a second crosslinker AI102.

3. The pH-responsive hydrogel biocarrier according to claim 1, wherein the methacrylated hyaluronic acid is synthesized from esterification of sodium hyaluronate and methacrylic anhydride in a molar ratio of 1:30.

4. The pH-responsive hydrogel biocarrier according to claim 2, wherein the molar ratio of the first crosslinker GDMA and the second crosslinker AI102 is (1-4):(0-2).

5. A method of preparing pH-responsive hydrogel biocarrier of any one of claims 1-3, wherein the said method comprises that methacrylated hyaluronic acid is initiated into a crosslinking reaction which is irradiated by 365 nm ultraviolet laser in the presence of photoinitiator, a first crosslinker GDMA and a second crosslinker AI102.

6. A pH-responsive hydrogel biocarrier loaded with therapeutic protein according to any one of claims 1-3, wherein the pH-responsive hydrogel biocarrier loaded with therapeutic protein is prepared by freeze-drying the hydrogel biocarrier and loading the therapeutic protein into the pH-responsive hydrogel biocarrier by dry immersion method.

7. The pH-responsive hydrogel biocarrier loaded with therapeutic protein according to claim 6, wherein the therapeutic protein is a platelet-derived growth factor.

8. The pH-responsive hydrogel biocarrier loaded with therapeutic protein according to claim 7, wherein the platelet-derived growth factor is in the form of BB type dimer.

9. A method of preparing the pH-responsive hydrogel biocarrier loaded with therapeutic protein of any one of claims 6-8, wherein the method comprises the steps of freeze-drying the hydrogel biocarrier, and load the therapeutic protein into the pH-responsive hydrogel biocarrier by dry immersion method.

10. The application of the pH-responsive hydrogel biocarrier loaded with therapeutic protein of any one of claims 6-8 in the preparation of drugs for promoting wound repair and tissue regeneration.
